(19) United States
(12) Patent Application Publication
(10) Pub. No.: US 2002/0063500 A1
(43) Pub. Date: May 30, 2002

(54) Miniature X-ray tube constructions

(75) Inventor: Hanan Keren, Kfar Saba (IL)

Correspondence Address: G.E. EHRLICH LTD., c/o ANTHONY CASTORINA, SUITE 207, 2001 JEFFERSON DAVIS HIGHWAY, ARLINGTON, VA, 22202, US

(73) Assignee: Medirad I.R.T. Ltd.

(21) Appl. No.: 09/827,918

(22) Filed: Apr. 9, 2001

(30) Foreign Application Priority Data
Nov. 30, 2000 (IL) 140025

(51) Int. Cl. H01J 7/26

(52) U.S. Cl. 313/30; 313/35

(57) ABSTRACT

An X-ray tube includes an evacuated envelope; a cold cathode mounted at one end of the envelope and capable of field emission of electrons when subjected to a high electrostatic field; and an anode mounted at the opposite end of the envelope coaxial with and axially spaced from the cathode, and capable of emitting X-rays when struck by electrons emitted by the cathode. The envelope includes an end wall made of thermally-conductive and electrically-insulating material in contact with the anode and formed with a fluid cooling channel to remove the heat generated at the anode. In one described embodiment, the cathode includes a body of a getter material which is both electron emissive and gas absorptive. In other described embodiments, the cathode includes a carbon nanotube field-emission electron source.

### FIELD AND BACKGROUND OF THE INVENTION

The present invention relates to X-ray tubes, and particularly to X-ray tubes of a miniature construction enabling them to be used in various medical applications, including treating restenosis in blood vessels, cancer therapy, imaging of small body parts, and the like. The invention is particularly useful in coronary revascularization therapy to remove vascular obstructions, and is therefore described below with respect to such application, but may also be used in many other medical applications like radiation therapy of cancers of various cancers as those in gastro-enterology, colorectal and intestines, gynecology and radiation therapy during surgery, as well as in various industrial applications such as in non-destructive testing of parts.

As described in my prior Patent Applications Ser. No. 09/414,853, filed Oct. 12, 1999, and Ser. No. 09/494,043 filed Jan. 31, 2000, the contents of which are hereby incorporated by reference, coronary vascular obstructions often require coronary bypass surgery, but frequently such an obstruction may be removed or reduced by Percutaneous Coronary Revascularization (PCR), such as by “balloon angioplasty”. However, such a PCR treatment frequently results in a renarrowing of the vessel, called “restenosis”, triggered by the injury to the vessel wall. Thus, the injury itself may trigger a healing response in the form of growth of a new inner lining within the vessel to heal the injured area (“intimal hyperplasia”).

Restenosis is commonly treated today by stenting. It has been found, however, that the placement of a stent may actually increase hyperplasia and thereby aggravate restenosis, rather than reduce it. Moreover, restenosis following stenting is particularly difficult to treat.

Another technique now being investigated for preventing renarrowing of the vessel caused by hyperplasia is by the use of drugs, but this approach introduces other problems relating to the drug used.

At the present time, the application of radiation appears to be the most promising treatment now being examined for the prevention of restenosis following PCR. Radiation has been found to work particularly well in inhibiting new growth as has been shown for years in cancer management. However, external beam radiation, administered in relatively high doses, has a damaging effect on the patient's body, and therefore this approach does not appear to be suitable for reducing restenosis.

Using low-dosage radioactive sources, such as seeds temporarily implanted or inserted into the patient's body (endiovascular brachytherapy), has also been proposed as a treatment for the prevention of restenosis following PCR. U.S. Pat. No. 5,683,345, of Nov. 4, 1997, discloses a technique utilizing a catheter to deliver, to a desired site in the vascular system, a radiation source in the form of a plurality of individually sealed seeds of radioactive material emitting beta radiation. However, using such radioactive devices requires special storage, shipping, handling and disposal procedures that must be closely controlled and monitored to minimize the risks.

It has recently been proposed to use a non-radioactive source, in the form of a miniature X-ray emitter, for this purpose, as described in Chapter 14 of Handbook of Vascular Brachytherapy, edited by Ron Waksman and Patrick W. Serruys, published by Martin Dunitz Ltd., London, 1998. According to the brief description in this publication, the X-ray emitter consumes 1 watt of power, operates at 20 KV, and provides operational current of 50 uA. Presumably, the X-ray emitter was of the conventional construction in which the cathode is heated by a filament to emit the electrons striking the anode at the velocity required to produce the X-ray radiation therefrom. Thus, the article states “To provide the necessary cooling, a saline solution is continuously pumped through the sheath at a flow rate of 10-15 ml/min.” The power required, and the heat generated by such an X-ray emitter, would appear to significantly limit its suitability for use against restenosis following PCR.

U.S. Pat. No. 5,854,822, of Dec. 29, 1998 discloses a miniature X-ray device having a cold cathode composed of a material that also allows it to act as a getter. Specifically mentioned in that patent is a getter material including a sintered powder mixture of titanium and an alloy of vanadium, iron and zirconium which, in accordance with one embodiment, may also include diamond powder. As described in that patent, the heat generation may be such as to require cooling in some applications but not in other applications. U.S. Pat. No. 6,095,966 discloses a miniature X-ray device which includes a balloon through which fluid is circulated to dissipate the heat. However, the constructions illustrated in those patents would appear to present substantial difficulties in commercial production using existing vacuum-tube manufacturing technology.

The two above-cited patent applications, Ser. Nos. 09/414,853 and 09/494,043, disclose miniature X-ray tubes without external cooling. This limits the maximum tube output without excessive heating, which is a serious drawback in their use for medical applications. Other cold cathode miniature X-ray tube constructions are described in U.S. Pat. Nos. 3,714,486 and 5,729,583, but such constructions are also not particularly suitable for medical applications of the type described above.

### OBJECTS AND BRIEF SUMMARY OF THE INVENTION

One object of the present invention is to provide an X-ray tube of an improved miniaturized construction, particularly useful for many therapeutic and/or diagnostic medical applications, but which can also be used in many industrial applications, such as in the non-destructive testing of parts. Another object of the invention is to provide an X-ray tube of a construction which includes cooling to increase heat dissipation, but which still can be constructed in relatively miniaturized form for many therapeutic, diagnostic and industrial applications. A further object of the invention is to provide an X-ray tube, of a construction which includes a cold cathode providing a high-intensity source of field-emission electrons making the X-ray tube particularly suitable for medical applications.

According to one broad aspect of the present invention, there is provided an X-ray tube, comprising: an evacuated envelope; a cold cathode mounted at one end of the envelope and capable of field emission of electrons when subjected to a high electrostatic field; and an anode mounted at the opposite end of the envelope coaxial with and axially spaced from the cathode, and capable of emitting X-rays when struck by electrons emitted by the cathode; the envelope including at least one end wall made of thermally-conductive and electrically-insulating material in high heat-exchange relation with the anode and formed with a fluid cooling channel to remove the heat generated at the anode.

Such a construction, permitting external cooling of the tube, provides for a higher rate of heat dissipation, and thereby enables such miniaturized X-ray tubes to be used in many applications, particularly medical applications, requiring higher tube outputs without excessive heating.

According to further features in the described preferred embodiments, the end wall is made of a ceramic material, and the thermally-conductive and electrically-insulating material is in contact with a substantial portion of the anode except a face thereof facing the cathode.

In one described preferred embodiment, the cathode includes a body of a getter material which is both electron emissive and gas absorptive.

In a second described preferred embodiment, the cathode includes a carbon nanotube field-emission electron source. Such electron sources, and their methods of production, are described in the literature, for example in S. Iijimia, Nature (London) 354, 56 (1991); W. A. de Heer et al, Science, 270, 1179-1180 (1995); and Q. H. Wang et al., Appl. Phys. Lett. 70 (24), 3308-3310, Jun. 16, 1997, the contents of which are hereby incorporated by reference as if reproduced herein. The use of such field-emission electron sources in X-ray tubes of the foregoing type has been found to enable the X-ray tubes to be miniaturized such as to be useful in many medical applications requiring high tube outputs without excessive heating.

According to another aspect of the present invention, therefore, there is provided an X-ray tube, comprising: an evacuated envelope including a cylindrical sleeve of a ceramic material, a first end wall sealingly closing one end of the cylindrical sleeve, and a second end wall sealingly closing the opposite end of the cylindrical sleeve; a cold cathode mounted in one of the end walls and capable of field emission of electrons when subjected to a high electrostatic field; and an anode mounted in the other of the end walls coaxial with, and axially spaced from, the cathode, and capable of emitting X-rays when struck by electrons emitted by the cathode; the cathode including a carbon nanotube field-emission electron source.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a sectional view illustrating one X-ray tube construction in accordance with the present invention;

FIG. 2 is a side elevational view of the tube of FIG. 1 after the tubulation for evacuating the envelope has been removed;

FIG. 3 is a sectional view illustrating another X-ray tube construction in accordance with the present invention;

FIG. 4 is a side elevational view of the tube of FIG. 3 after the tubulation for evacuating the envelope has been removed; and

FIG. 5 is a side elevational view of a third X-ray tube construction similar to that of FIGS. 3 and 4 but without the fluid cooling feature.

### DESCRIPTION OF PREFERRED EMBODIMENTS

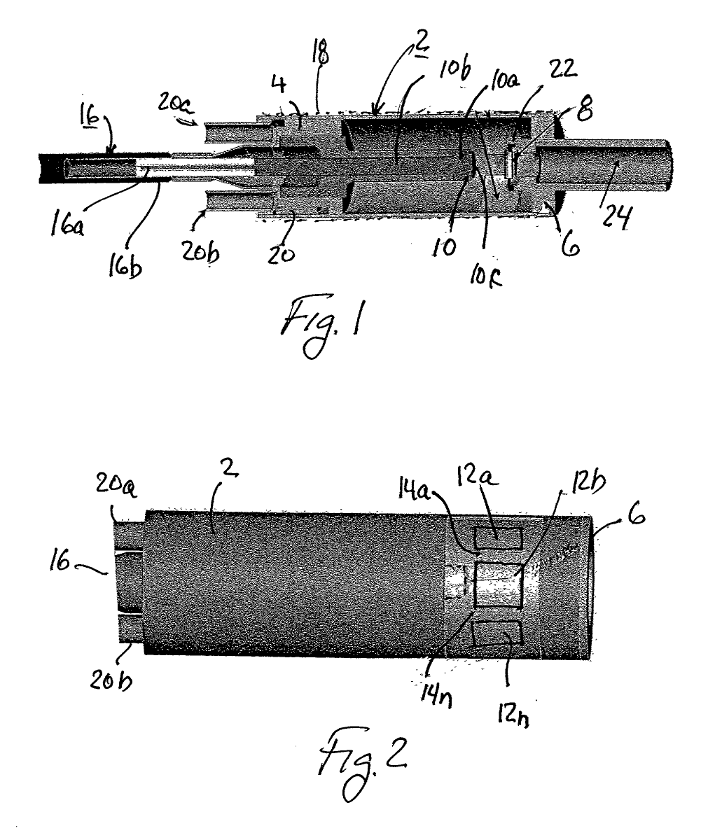

The X-ray Tube of FIGS. 1 and 2

The X-ray tube illustrated in FIG. 1 comprises an evacuated envelope constituted of a cylindrical sleeve 2 closed at one end by an end wall 4 sealingly bonded to the tube 2, and at the opposite end by another end wall 6 also sealingly bonded to the tube 2. Sleeve 2 and the two end walls 4, 6 are each made of ceramic material. The ceramic material used for end wall 4 is thermally-conductive, and that used for end wall 6 is electrically-conductive, as will be described more particularly below.

Ceramic end wall 6 mounts a cold cathode 8 which is capable of field emission of electrons when subjected to a high electrostatic field. Ceramic end wall 4 mounts an anode 10 coaxial with, and axially spaced from, the cathode 8. Anode 10 is made of a material, such as tungsten, capable of emitting X-rays when struck by electrons emitted by the cathode.

As shown in FIG. 2, envelope 2 includes a plurality of X-ray transparent windows 12a, 12b - - - 12n radially aligned with the space between the anode 10 and cathode 8, and circumferentially spaced from each other by a plurality of axially extending struts 14a - - - 14n.

The anode 10 is of cylindrical configuration and extends through end wall 4 for connection to the inner conductor 16a of an electrical cable 16 for applying a high voltage between the anode and the cathode 8. Electrical cable 16 is of a flexible construction and includes an outer conductor 16b in the form of a braid electrically connected to the cathode by an electrically-conductive coating 18 applied to the outer surface of envelope 2 including its end walls 4, 6, as described in the above-cited patent applications.

Most of the heat generated during the operation of the X-ray tube is concentrated in the anode, and particularly in the region where it is electrically connected to cable 16. For this reason, the outer surface of this region of the anode is in direct contact with the ceramic material of end wall 4, which ceramic material, as indicated earlier, must therefore be thermally-conductive, as well electrically insulating. End wall 4 is formed with a fluid cooling channel 20 having an inlet 20a and an outlet 20b for removing the heat generated at the anode.

As further shown in FIG. 1, cylindrical anode 10 is of larger diameter at the end thereof 10a facing the cathode 8, and is of reduced diameter for the remaining length of the anode as shown at 10b. As also seen in FIG. 1, the ceramic material of end wall 4 covers the complete outer surface of portion 10b of the anode, but leaves the outer surface of the larger diameter portion 10a, and also the end face 10c of the anode, bare to expose these surfaces directly to the electrons emitted from the cathode. Such a construction produces a maximum surface area of the anode to be struck by the electrons from the cathode 8 for the generation of X-rays, while maximizing the dissipation of the heat generated in the anode.

In the production of the tube 2, the anode 10 is mounted to the ceramic end wall 4, previously formed with the fluid cooling channel 20. End wall 4 is then mounted within the respective end of cylindrical sleeve 2 and sealingly bonded thereto. The cathode 8 is also preferably mounted in end wall 6 before that end wall is received within and sealingly bonded to the opposite end of the cylindrical sleeve 2.

Cathode 8 may be mounted at any suitable manner, such as by the provision of a plurality of radially-extending struts 22. As clearly seen in FIG. 1, the cathode is of disc configuration and is received within a recess of complimentary configuration formed in the inner face of the tube end wall 6. In the construction illustrated in FIGS. 1 and 2, cathode 8 preferably is constituted of, or includes, a getter material which is both electron emissive and gas absorptive, as described, for example, in the above-cited pending applications incorporated herein by reference.

End wall 6 is made of an electrically-conductive ceramic material to provide electrical continuity to the inner conductor 16a of the electrical cable 16. End wall 6 also preferably includes the tubulation 24 used for evacuating the envelope, which tubulation is sealed closed after the envelope has been evacuated. After the envelope has thus been sealed, the inner conductor 16a of the electrical cable 16 is connected to the anode 8. A metal spray coating may then be applied to the outer braid 16b of the electrical cable, and to the outer face of the envelope 2, including its end walls 4, 6, except for the windows 12a - - - 12n, to provide the electrically-conductive coating 18 electrically connecting the cathode 8 to the outer braid of the electrical cable 16.

As one example, the X-ray tube illustrated in FIGS. 1 and 2 could be 0.12-0.36 inches in diameter; the tube length could be 0.45-1.35 inches, and the voltage could be between 10-40 kV, preferably about 20 kV. When working in a pulsed mode with a tube voltage of 10-40 kV, the tube dimensions can be reduced to about one-half of the above, namely to a tube diameter of 0.06 inches and a tube length of 0.2 inches.

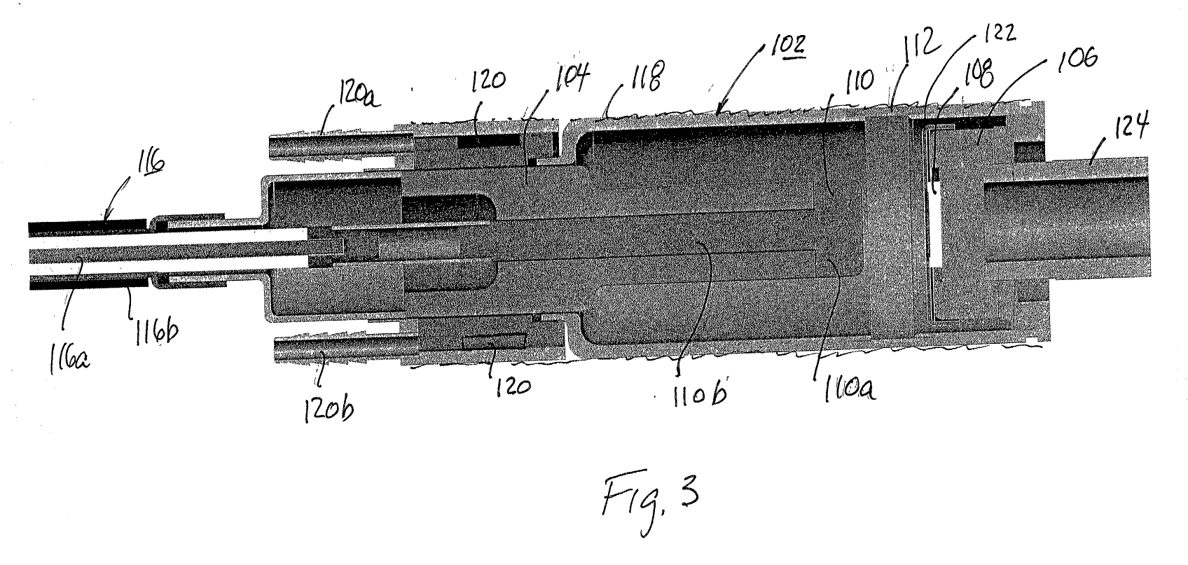

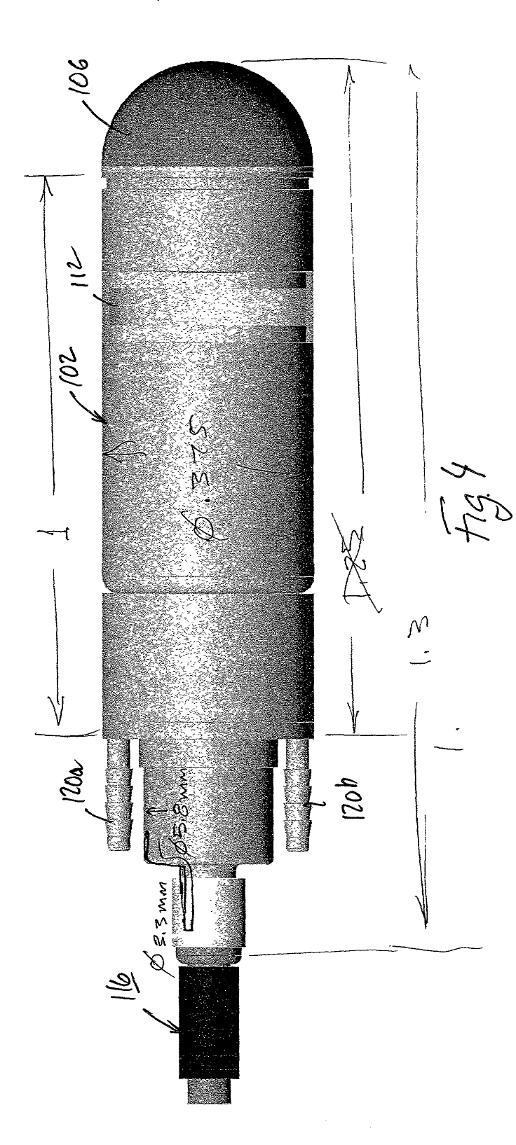

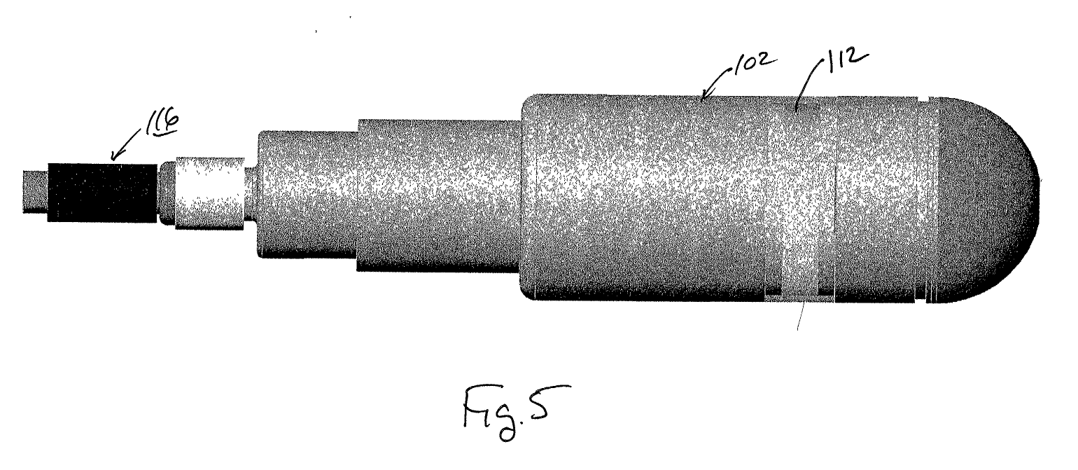

The X-ray Tube of FIGS. 3 and 4

The X-ray tube of FIGS. 3 and 4 is of a very similar construction as that in FIGS. 1 and 2. One important difference is that, instead of using a cathode including a body of a getter material which is both electron emissive and gas absorptive as described in the above-cited pending patent applications incorporated herein by reference, there is used, instead, a cold cathode including a carbon nanotube field-emission electron source, such as described in the above-cited publications also incorporated herein by reference. Another difference is that, instead of providing the envelope with a plurality of X-ray transparent windows radially aligned with the space between the anode and the cathode and circumferentially spaced from each other, there is provided instead a single X-ray transparent window of annular configuration radially aligned with the space between the anode and the cathode.

In order to facilitate understanding, the parts of the X-ray tube illustrated in FIGS. 3 and 4 which generally correspond to the parts described above in the X-ray tube of FIGS. 1 and 2 are identified by the same reference numerals, but increased by “100”.

Thus, the X-ray tube illustrated in FIGS. 3 and 4 also comprises an evacuated envelope constituted of a cylindrical sleeve 102 closed at one end by an end wall 104 sealingly bonded to the tube 102, and at the opposite end by another end wall 106 also sealingly bonded to the tube. Sleeve 102, and the two end walls 104, 106, are each made of electrically insulating ceramic material. The ceramic material used for end wall 104 is highly thermally-conductive; and that used for end wall 106 is electrically-conductive, as in the X-ray tube of FIGS. 1 and 2.

Ceramic end wall 106 mounts a cold cathode 108 which is capable of field emission of electrons when subjected to a high electrostatic field. Ceramic end wall 104 mounts an anode 110 coaxial with, and axially spaced from, the cathode 108, and is made of a material, such as tungsten, capable of emitting X-rays when struck by electrons emitted by the cathode.

As indicated earlier, whereas the cold cathode 8 in the tube construction of FIGS. 1 and 2 is constituted of, or includes, a getter material which is both electron emissive and gas absorptive as described for example in the above-cited pending applications incorporated herein by reference, the cold cathode 108 in the tube of FIGS. 3 and 4 is constituted of, or includes, a carbon nanotube field-emission electron source such as described in the above-cited publications.

Envelope 102 is formed with an X-ray transparent window 112 of annular configuration radially aligned with a space between the anode 110 and the cathode 108. Window 112 may be, for example, of beryllium.

The anode 110 is of cylindrical configuration and extends through end wall 104 for connection to the inner conductor 116a of an electrical cable 116, for applying a high voltage between the anode and the cathode 108. Electrical cable 116 is of a flexible construction and includes an outer conductor 116b in the form of a braid electrically connected to the cathode 110 by an electrically-conductive coating 118 applied to the outer surface of an envelope 102, including its end walls 104 and 106, as in the FIGS. 1, 2 construction.

As also in FIGS. 1, 2 construction, cylindrical anodes 110 is of larger diameter at the end thereof 110a facing the cathode 108, and is of reduced diameter for its remaining length 110b. The ceramic material of end wall 104 covers the complete outer surface of anode portion 110b, but leaves the outer surface of the larger-diameter portion 110a, and also the end face 110c, bare to expose these surfaces directly to the electrons emitted from the cathode. End wall 104 is formed with a fluid cooling channel 120 having an inlet 120a, and an outlet 120b, for removing the heat generated at the anode.

Cathode 108 is of disc configuration, and is received within a recess of complementary configuration formed in the inner face of the tube end wall 106. The cathode may be mounted in any suitable manner within the end wall, such as by the provision of a plurality of radially-extending struts 122. As described earlier, cathode 108 is made of, or includes, a carbon nanotube construction as described in the above-cited publications to serve as a high-intensity source of field-emission electrons.

End wall 106 includes the tubulation 124 used for evacuating the envelope, which tubulation is sealed closed after the envelope has been evacuated. After the envelope has thus been sealed, the inner conductor 116a of the electrical cable 116 is connected to the anode 108. A metal spray coating 118 is then applied to the outer braid 116b of the electrical cable, and to the outer face of the envelope 102, including its end walls 104, 106, to electrically connect the cathode 108 to the outer braid 116b of the cable.

As indicated above, the X-ray tube illustrated in FIGS. 3 and 4, including the carbon nanotube cold cathode enables the X-ray tube to be of a miniaturized construction for use, particularly in medical applications, requiring relatively high tube outputs without excessive heating.

The X-ray Tube of FIG. 5

FIG. 5 illustrates an X-ray tube of the same construction as in FIGS. 3 and 4, except that the fluid cooling channel 120, its inlet 120a and its outlet 120b, are omitted. Such an X-ray tube construction may therefore be suitable in applications, particularly medical applications, requiring lower tube outputs, or tolerating more heat-generation, than the X-ray tube of FIGS. 3 and 4. Since the construction of the X-ray tube illustrated in FIG. 5 is otherwise the same as described above with respect to FIGS. 3 and 4, the same reference numerals have been used to identify corresponding parts to facilitate understanding.

While the invention has been described with respect to several preferred embodiments, it will be appreciated that these are set forth merely for purposes of example, and that many variations, modifications and other applications of the invention may be made.

What is claimed is:

1. An X-ray tube, comprising:
an evacuated envelope;
a cold cathode mounted at one end of said envelope and capable of field emission of electrons when subjected to a high electrostatic field;
and an anode mounted at the opposite end of said envelope coaxial with and axially spaced from said cathode, and capable of emitting X-rays when struck by electrons emitted by the cathode;
said envelope including at least one end wall made of a thermally-conductive and electrically-insulating material in high heat-exchange relation with the anode and formed with a fluid cooling channel to remove the heat generated at said anode.

2. The X-ray tube according to claim 1, wherein said end wall is made of a ceramic material.

3. The X-ray tube according to claim 1, wherein said thermally-conductive and electrically-insulating material is in contact with a substantial portion of the anode except a face thereof facing the cathode.

4. The X-ray tube according to claim 1, said thermally-conductive and electrically-insulating material covers substantially the complete outer surface of the anode except an end face of the anode facing the cathode and the outer surface of the anode adjacent to said end face, said end face and outer surface adjacent thereto being bare to expose them directly to the electrons emitted from the cathode.

5. The X-ray tube according to claim 4, wherein the anode is of cylindrical configuration, and said bare outer surface of the anode is of larger outer diameter than the remainder of the anode, said remainder of the anode being covered by said thermally-conductive and electrically-insulating material.

6. The X-ray tube according to claim 1, wherein said envelope is made of a ceramic material and includes at least one X-ray transparent window radially aligned with the space between the anode and the cathode.

7. The X-ray tube according to claim 6, wherein said envelope includes a plurality of X-ray transparent windows radially aligned with the space between the anode and the cathode and circumferentially spaced from each other.

8. The X-ray tube according to claim 1, wherein said envelope includes a cylindrical sleeve, with said one end wall formed with said fluid cooling channel sealingly closing one end of the cylindrical sleeve, and with an opposite end wall sealingly closing the opposite end of the cylindrical sleeve.

9. The X-ray tube according to claim 8, wherein said envelope, said one end wall, and said opposite end wall, are all made of a ceramic material.

10. The X-ray tube according to claim 8, wherein said one end wall mounts said anode.

11. The X-ray tube according to claim 10, wherein said opposite end wall mounts said cathode.

12. The X-ray tube according to claim 11, wherein said opposite end wall also includes a tubulation used for evacuating the envelope and closed after the envelope is evacuated.

13. The X-ray tube according to claim 11, wherein said one end wall also includes a cable having a center conductor connected to said anode, and an outer conductor connected to the cathode via an electrically-conducting coating applied to the outer surface of said envelope and its end walls.

14. The X-ray tube according to claim 11, wherein said cathode includes a body of a getter material which is both electron emissive and gas absorptive.

15. The X-ray tube according to claim 11, wherein said cathode includes a carbon nanotube field-emission electron source.

16. An X-ray tube, comprising:
an evacuated envelope including a cylindrical sleeve of a ceramic material, a first end wall sealingly closing one end of said cylindrical sleeve, and a second end wall sealingly closing the opposite end of said cylindrical sleeve;
a cold cathode mounted in one of said end walls and capable of field emission of electrons when subjected to a high electrostatic field;
and an anode mounted in the other of said end walls coaxial with, and axially spaced from, said cathode, and capable of emitting X-rays when struck by electrons emitted by the cathode;
said cathode including a carbon nanotube field-emission electron source.

17. The X-ray tube according to claim 16, wherein said anode is of a cylindrical configuration, and said ceramic material is thermally-conductive and electrically-insulating and covers substantially the complete outer surface of the anode except an end face thereof facing the cathode and the outer surface of the anode adjacent to said end face, said end face and outer surface adjacent thereto being bare to expose them directly to the electrons emitted from the cathode.

18. The X-ray tube according to claim 17, wherein said bare outer surface of the anode is of larger outer diameter than the remainder of the anode covered by said thermally-conductive and electrically-insulating material.

19. The X-ray tube according to claim 16, wherein said envelope includes an X-ray transparent window of annular configuration radially aligned with the space between the anode and the cathode.

20. The X-ray tube according to claim 16, wherein said end wall mounting the anode is made of a thermally-conductive, electrically-insulating material in heat-exchange relation with the anode and formed with a fluid cooling channel to remove heat generated at said anode; and said thermally-conductive electrically-insulating material is in contact with a substantial portion of the anode except a face thereof facing the cathode.
